(19) United States
(12) Patent Application Publication
(10) Pub. No.: US 2012/0010445 A1
(43) Pub. Date: Jan. 12, 2012

(54) Low Energy Alcohol Recovery Processes

(75) Inventors: Victor JOHNSTON, Houston, TX (US); David LEE, Seabrook, TX (US); Adam OROSCO, Houston, TX (US); Lincoln SARAGER, Houston, TX (US); Trinity HORTON, Houston, TX (US); Radmila JEVTIC, Pasadena, TX (US)

(73) Assignee: CELANESE INTERNATIONAL CORPORATION, Dallas, TX (US)

(21) Appl. No.: 13/094,691

(22) Filed: Apr. 26, 2011

Related U.S. Application Data
(60) Provisional application No. 61/363,089, filed on Jul. 9, 2010.

(51) Int. Cl. C07C 29/80; C07C 29/76

(52) U.S. Cl. 568/885

(57) ABSTRACT

Recovery of ethanol from a crude ethanol product obtained from the hydrogenation of acetic acid using various combinations of membranes and/or distillation columns.

### CROSS REFERENCE TO RELATED APPLICATIONS

This application claims priority to U.S. Provisional App. No. 61/363,089, filed on Jul. 9, 2010, the entirety of which is incorporated herein by reference.

### FIELD OF THE INVENTION

The present invention relates generally to processes for producing ethanol and, in particular, to a low energy process for recovering ethanol using membranes.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulose materials, such as corn or sugar cane. Conventional methods for producing ethanol from petrochemical feed stocks, as well as from cellulose materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulose material, are converted to ethanol by fermentation. However, fermentation is typically used for consumer production of ethanol, which is suitable for fuels or human consumption. In addition, fermentation of starchy or cellulose materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. During the reduction of alkanoic acid, e.g., acetic acid, other compounds are formed with ethanol or are formed in side reactions. These impurities limit the production and recovery of ethanol from such reaction mixtures. For example, during hydrogenation, esters are produced that together with ethanol and/or water form azeotropes, which are difficult to separate. In addition when conversion is incomplete, unreacted acid remains in the crude ethanol product, which must be removed to recover ethanol.

EP02060553 describes a process for converting hydrocarbons to ethanol involving converting the hydrocarbons to ethanoic acid and hydrogenating the ethanoic acid to ethanol. The stream from the hydrogenation reactor is separated to obtain an ethanol stream and a stream of acetic acid and ethyl acetate, which is recycled to the hydrogenation reactor.

Ethanol recovery systems for other types of ethanol production processes are also known. For example, U.S. Pub. No. 2008/0207959 describes a process for separating water from ethanol using a gas separation membrane unit. The gas separation membrane unit may be used to remove water from a fermentation broth that has been partially dewatered, for example by one or more of a distillation column or molecular sieves. Additional systems employing membranes and distillation columns are described in U.S. Pat. Nos. 7,732,173; 7,594,981; and 4,774,365, the entireties of which are incorporated herein by reference. See also Huang, et al, “Low-Energy Distillation-Membrane Separation Process,” Ind. Eng. Chem. Res., Vol. 40 (2010), pg. 3760-68, the entirety of which is incorporated herein by reference.

The need remains for improved processes for recovering ethanol from a crude product obtained by reducing alkanoic acids, such as acetic acid, and/or other carbonyl group-containing compounds.

### SUMMARY OF THE INVENTION

In a first embodiment, the present invention is directed to a process for producing ethanol, comprising the steps of hydrogenating acetic acid in a reactor in the presence of a catalyst to form a crude ethanol product comprising ethanol, acetic acid and water; separating at least a portion of the crude ethanol product in a distillation column into a distillate comprising ethanol and water, and a residue comprising acetic acid and water; and passing at least a portion of the distillate stream to one or more membranes to yield an ethanol stream and a water stream.

In a second embodiment, the present invention is directed to a process for producing ethanol, comprising the steps of hydrogenating acetic acid in a reactor in the presence of a catalyst to form a crude ethanol product comprising ethanol, ethyl acetate, and acetic acid; separating at least a portion of the crude ethanol product in a distillation column into a distillate comprising ethanol and ethyl acetate, and a residue comprising acetic acid; and passing at least a portion of the distillate stream to one or more membranes to yield an ethanol stream and an ethyl acetate stream.

In a third embodiment, the present invention is directed to a process for producing ethanol, comprising the steps of hydrogenating acetic acid in a reactor in the presence of a catalyst to form a crude ethanol product comprising ethanol, ethyl acetate, water, and acetic acid; separating at least a portion of the crude ethanol product in a first distillation column into a first distillate comprising ethanol, ethyl acetate, and water, and a first residue comprising acetic acid; and separating at least a portion of the first distillate in a second distillation column into a second distillate comprising ethyl acetate, and a second residue comprising ethanol and water; and passing at least a portion of the second residue to one or more membranes to yield an ethanol stream and an water stream.

In a fourth embodiment, the present invention is directed to a process for producing ethanol, comprising the steps of hydrogenating acetic acid in a reactor in the presence of a catalyst to form a crude ethanol product, comprising ethanol, acetic acid, and water; passing at least a portion of the crude ethanol product to a first membrane to separate a first permeate stream comprising acetic acid and a first retentate stream comprising ethanol and water; passing the first retentate stream to a second membrane to separate a second permeate stream comprising water and a second retentate stream comprising ethanol.

In a fifth embodiment, the present invention is directed to a process for producing ethanol, comprising the steps of hydrogenating acetic acid in a reactor in the presence of a catalyst to form a crude ethanol product, separating at least a portion of the crude ethanol product in at least one distillation column to form a derivative stream, and passing at least a portion of the derivative stream to at least one membrane to separate a stream comprising ethanol.

In a sixth embodiment, the present invention is directed to a process for producing ethanol, comprising the steps of hydrogenating acetic acid in a reactor in the presence of a catalyst to form a crude ethanol product, passing at least a portion of the crude ethanol product to at least one membrane to separate at least one stream, and separating at least a portion of the at least one stream in at least one membrane distillation column to form a derivative stream comprising ethanol.

In a seventh embodiment, the present invention is directed to a process for producing ethanol, comprising the steps of hydrogenating acetic acid in a reactor in the presence of a catalyst to form a crude ethanol product, and passing at least a portion of the crude ethanol product to at least one membrane to separate a stream comprising ethanol.

In an eighth embodiment, the present invention is directed to a process for producing ethanol, comprising the steps of providing a crude ethanol product comprising ethanol and water, wherein the ethanol is in an amount of from 15 wt. % to 70 wt. %, separating at least a portion of the crude ethanol product in at least one distillation column to form a derivative stream, and passing at least a portion of the derivative stream to at least one membrane to separate a stream comprising ethanol.

In a ninth embodiment, the present invention is directed to a process for producing ethanol, comprising the steps of providing a crude ethanol product comprising ethanol and water, wherein the ethanol is in an amount of from 15 wt. % to 70 wt. %, passing at least a portion of the crude ethanol product to at least one membrane to separate a retentate stream, and separating at least a portion of the retentate stream in at least one membrane distillation column to form a derivative stream comprising ethanol.

In a tenth embodiment, the present invention is directed to a process for producing ethanol, comprising the steps of providing a crude ethanol product comprising ethanol and water, wherein the ethanol is in an amount of from 15 wt. % to 70 wt. %, and passing at least a portion of the crude ethanol product to at least one membrane to separate a retentate stream comprising ethanol.

In an eleventh embodiment, the present invention is directed to a process for producing ethanol, comprising the steps of hydrogenating acetic acid in a reactor in the presence of a catalyst to form a crude ethanol product comprising ethanol and water, separating at least a portion of the crude ethanol product in a distillation column into a distillate comprising ethanol and water, and a residue comprising water, passing at least a portion of the distillate to a first membrane to separate a first permeate comprising water, and a first retentate stream comprising ethanol and water, and passing at least a portion of the first retentate to a second membrane to separate a second permeate comprising water and ethanol, and a second retentate stream comprising a finished ethanol product.

In a twelve embodiment, the present invention is directed to a process for producing ethanol, comprising the steps of providing a crude ethanol product comprising ethanol and water, wherein the ethanol is in an amount of from 15 wt. % to 70 wt. %, separating at least a portion of the crude ethanol product in a distillation column into a distillate comprising ethanol and water, and a residue comprising water, passing at least a portion of the distillate to a first membrane to separate a first permeate comprising water, and a first retentate stream comprising ethanol and water, and passing at least a portion of the first retentate to a second membrane to separate a second permeate comprising water and ethanol, and a second retentate stream comprising a finished ethanol product.

### BRIEF DESCRIPTION OF DRAWINGS

The invention may be more completely understood in consideration of the following detailed description of various embodiments of the invention in connection with the accompanying drawings, wherein like numerals designate similar parts.

FIG. 1 is a schematic diagram of an ethanol production system having a combined distillation and membrane separation system in accordance with one embodiment of the present invention.

FIG. 2 is a schematic diagram of an ethanol production system having a combined distillation and membrane separation system with two distillation columns in accordance with one embodiment of the present invention.

FIG. 3A is a schematic diagram of an ethanol production system having a combined distillation and membrane separation system with three distillation columns in accordance with one embodiment of the present invention.

FIG. 3B is a schematic diagram of an ethanol production system having a membrane separation system within a three distillation columns in accordance with one embodiment of the present invention.

FIG. 4 is a schematic diagram of an ethanol production system having a combined distillation and membrane separation system with two distillation columns in accordance with one embodiment of the present invention.

FIG. 5 is a schematic diagram of an ethanol production system having a combined distillation and membrane separation system with a weak acid recovery zone in accordance with one embodiment of the present invention.

FIG. 6 is a membrane for separating the crude ethanol product in accordance with one embodiment of the present invention.

FIG. 7 is a schematic diagram of an ethanol production system having a combined distillation and membrane separation system with one distillation column in accordance with one embodiment of the present invention.

FIG. 8 is a schematic diagram of an ethanol production system having a combined distillation and membrane separation system with one distillation column in accordance with one embodiment of the present invention.

FIG. 9 is a schematic diagram of an ethanol production system having a combined distillation and membrane separation system with two distillation columns in accordance with one embodiment of the present invention.

FIG. 10 is a schematic diagram of an ethanol production system having a membrane separation system in accordance with one embodiment of the present invention.

FIG. 11 is a schematic diagram of an ethanol production system having a membrane separation system in accordance with another embodiment of the present invention.

### DETAILED DESCRIPTION OF THE INVENTION

### Introduction

The present invention relates generally to low energy ethanol separation processes for producing ethanol. The processes of the present invention may be applied to a variety of ethanol production systems and beneficially may be used in applications for the recovery and/or purification of ethanol on an industrial scale. For example, various aspects of the present invention relate to processes for recovering and/or purifying ethanol produced by a process comprising hydrogenating acetic acid in the presence of a catalyst. The hydrogenation reaction produces a crude ethanol product that comprises ethanol, water, ethyl acetate, acetic acid, and other impurities.

Crude product streams containing multiple different species typically are purified using a series of distillation columns. Depending on their operating parameters, however, distillation columns can consume a significant amount of energy. In some embodiments, the present invention relates to the use of one or more membranes in combination with one or more separation columns, e.g., distillation columns, to separate ethanol from a crude ethanol product. In some aspects, for example, the membranes beneficially may eliminate the necessity for one or more separation columns. Since membranes typically require less energy than distillation columns, the present invention provides lower energy processes for recovering ethanol from a crude ethanol product.

The membranes of the present invention are preferably pervaporation membranes. Suitable membranes include shell and tube membrane modules having one or more porous material elements therein. Non-porous material elements may also be included. The material elements may include polymeric element such as polyvinyl alcohol, cellulose esters, and perfluoropolymers. Membranes that may be employed in embodiments of the present invention include those described in Baker, et al., “Membrane separation systems: recent developments and future directions,” (1991) pages 151-169, Perry et al., “Perry's Chemical Engineer's Handbook,” 7th ed. (1997), pages 22-37 to 22-69, the entireties of which are incorporated herein by reference.

In some embodiments, the crude ethanol product or a derivative stream thereof is fed to a membrane or an array of membranes. Derivative stream refers to any stream having components that originated in the crude ethanol product. For example, the derivative stream may be the distillate or residue obtained from separating the crude ethanol product in a distillation column.

Ethanol and water form an azeotrope that limits the recoverable ethanol in distillation columns to an ethanol product comprising about 92-96 wt. % ethanol. The use of one or more membranes according to the invention may advantageously provide the ability to “break” azeotropes without the use of entrains. The processes of the invention are preferably suited for recovering an ethanol product, such as an anhydrous ethanol product, having an ethanol concentration greater than the azeotrope ethanol concentration, preferably providing an ethanol concentration of at least 96 wt. % ethanol or greater or at least 99 wt. % or greater. In one embodiment, the crude ethanol product has few components other than ethanol and water, which allows more efficient ethanol recovery using membranes. Any other organic components, if present, in the crude ethanol product may stay with the ethanol instead of passing through the membranes with the water. For example, when ethyl acetate is present in the crude ethanol product in addition to ethanol and water, water preferably permeates the membrane while the ethanol and ethyl acetate are separated from the water together in the retentate.

In addition to ethanol and water, membranes also may be used to remove other components from the crude ethanol product. In one embodiment, for example, a hydrogen membrane may be used to remove hydrogen from the crude ethanol product. In another embodiment, a derivative stream of the crude ethanol product containing ethanol and ethyl acetate, but preferably little if any water, may be separated with a membrane to recover ethanol either as the permeate or the retentate stream depending on the membrane that is used. In addition, water membranes may also be used to separate water from the crude ethanol product and/or acid streams. Combinations of these membranes to separate different streams may be arranged to ultimately recover ethanol.

Distillation columns may also be used in combination with membranes to remove some of the components, such as acetic acid, ethyl acetate and acetaldehyde before or after passing the resulting derivative stream of the crude ethanol product through the one or more membranes. Optionally, the components, either in the permeate or retentate, may be removed in one or more distillation columns after passing through the membranes.

### Hydrogenation of Acetic Acid

The separation steps of the present invention may be used with any hydrogenation process to produce ethanol, but preferably is used with hydrogenation of acetic acid. The materials, catalysts, reaction conditions, and separation processes that may be used in the hydrogenation of acetic acid are described further below.

The raw materials, acetic acid and hydrogen, used in connection with the process of this invention may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259 and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from alternate carbon sources have drawn increasing interest. In particular, when petroleum is relatively expensive, it may become advantageous to produce acetic acid from synthesis gas (“syngas”) that is derived from more available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolyzed with additional natural gas to form synthesis gas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into synthesis gas, and U.S. Pat. No. 6,685,754, which discloses a method for the production of a hydrogen-containing gas composition, such as a synthesis gas including hydrogen and carbon monoxide, are incorporated herein by reference in their entireties.

The acetic acid fed to the hydrogenation reaction may also comprise other carboxylic acids and anhydrides, as well as acetaldehyde and acetone. Preferably, a suitable acetic acid feed stream comprises one or more of the compounds selected from the group consisting of acetic acid, acetic anhydride, acetaldehyde, ethyl acetate, and mixtures thereof. These other compounds may also be hydrogenated in the processes of the present invention. In some embodiments, the presence of carboxylic acids, such as propanoic acid or its anhydride, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the ethanol synthesis reaction zones of the present invention without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

Some embodiments of the process of hydrogenating acetic acid to form ethanol may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles.

The hydrogenation reaction may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 1500 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges, the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 12:1 to 1:1. Most preferably, the molar ratio of hydrogen to acetic acid is greater than 2:1, e.g., greater than 4:1 or greater than 8:1.

Contact or residence time can also vary widely, depending upon such variables as amount of acetic acid, catalyst, reactor, temperature, and pressure. Typical contact times range from a fraction of a second to more than several hours when a catalyst system other than a fixed bed is used, with preferred contact times, at least for vapor phase reactions, of from 0.1 to 100 seconds, e.g., from 0.3 to 80 seconds or from 0.4 to 30 seconds.

The hydrogenation of acetic acid to form ethanol is preferably conducted in the presence of a hydrogenation catalyst. Suitable hydrogenation catalysts include catalysts comprising a first metal and optionally one or more of a second metal, a third metal or any number of additional metals, optionally on a catalyst support. The first and optional second and third metals may be selected from Group IB, IIB, IIIB, IVB, VB, VIIB, VIIB, VIII transition metals, a lanthanide metal, an actinide metal or a metal selected from any of Groups IIIA, IVA, VA, and VIA. Preferred metal combinations for some exemplary catalyst compositions include platinum/tin, platinum/ruthenium, platinum/rhenium, palladium/ruthenium, palladium/rhenium, cobalt/palladium, cobalt/platinum, cobalt/chromium, cobalt/ruthenium, silver/palladium, copper/palladium, nickel/palladium, gold/palladium, ruthenium/rhenium, ruthenium/iron, copper/zinc, and cobalt/tin. Exemplary catalysts are further described in U.S. Pat. No. 7,608,744 and U.S. Pub. No. 2010/0029995, the entireties of which are incorporated herein by reference.

In another embodiment, the catalyst comprises a Co/Mo/S catalyst of the type described in U.S. Pub. No. 2009/0069609, the entirety of which is incorporated herein by reference.

In one embodiment, the catalyst comprises a first metal selected from the group consisting of copper, iron, cobalt, nickel, ruthenium, rhodium, palladium, osmium, iridium, platinum, titanium, zinc, chromium, rhenium, molybdenum, and tungsten. Preferably, the first metal is selected from the group consisting of platinum, palladium, cobalt, nickel, and ruthenium. More preferably, the first metal is selected from platinum and palladium. In embodiments of the invention where the first metal comprises platinum, it is preferred that the catalyst comprises platinum in an amount less than 5 wt. %, e.g., less than 3 wt. % or less than 1 wt. %, due to the high commercial demand for platinum.

As indicated above, in some embodiments, the catalyst further comprises a second metal, which typically would function as a promoter. If present, the second metal preferably is selected from the group consisting of copper, molybdenum, tin, chromium, iron, cobalt, vanadium, tungsten, palladium, platinum, lanthanum, cerium, manganese, ruthenium, rhenium, gold, and nickel. More preferably, the second metal is selected from the group consisting of copper, tin, cobalt, rhenium, and nickel. More preferably, the second metal is selected from tin and rhenium.

In certain embodiments where the catalyst includes two or more metals, e.g., a first metal and a second metal, the first metal is present in the catalyst in an amount from 0.1 to 10 wt. %, e.g., from 0.1 to 5 wt. %, or from 0.1 to 3 wt. %. The second metal preferably is present in an amount from 0.1 to 20 wt. %, e.g., from 0.1 to 10 wt. %, or from 0.1 to 5 wt. %. For catalysts comprising two or more metals, the two or more metals may be alloyed with one another or may comprise a non-alloyed metal solution or mixture.

The preferred metal ratios may vary depending on the metals used in the catalyst. In some exemplary embodiments, the mole ratio of the first metal to the second metal is from 10:1 to 1:10, e.g., from 4:1 to 1:4, from 2:1 to 1:2, from 1.5:1 to 1:1.5 or from 1.1:1 to 1:1.1.

The catalyst may also comprise a third metal selected from any of the metals listed above in connection with the first or second metal, so long as the third metal is different from the first and second metals. In preferred aspects, the third metal is selected from the group consisting of cobalt, palladium, ruthenium, copper, zinc, platinum, tin, and rhenium. More preferably, the third metal is selected from cobalt, palladium, and ruthenium. When present, the total weight of the third metal preferably is from 0.05 to 4 wt. %, e.g., from 0.1 to 3 wt. %, or from 0.1 to 2 wt. %.

In addition to one or more metals, in some embodiments of the present invention, the exemplary catalysts further comprise a support or a modified support. As used herein, the term “modified support” refers to a support material and a support material and a support modifier, which adjusts the acidity of the support material.

The total weight of the support or modified support, based on the total weight of the catalyst, preferably is from 75 to 99.9 wt. %, e.g., from 78 to 97 wt. %, or from 80 to 95 wt. %. In preferred embodiments that utilized a modified support, the support modifier is present in an amount from 0.1 to 50 wt. %, e.g., from 0.2 to 25 wt. %, from 0.5 to 15 wt. %, or from 1 to 8 wt. %, based on the total weight of the catalyst. The metals of the catalysts may be dispersed throughout the support, layered throughout the support, coated on the outer of the support (i.e., egg shell), or decorated on the surface of the support.

As will be appreciated by those of ordinary skill in the art, support materials are selected such that the catalyst system is suitably active, selective and robust under the process conditions employed for the formation of ethanol

Suitable support materials may include, for example, stable metal oxide-based supports or ceramic-based supports. Preferred supports include siliceous supports, such as silica, silica/alumina, a Group IIA silicate such as calcium metasilicate, pyrogenic silica, high purity silica, and mixtures thereof. Other supports may include, but are not limited to, iron oxide, alumina, titania, zirconia, magnesium oxide, carbon, graphite, high surface area graphitized carbon, activated carbons, and mixtures thereof.

As indicated, the catalyst support may be modified with a support modifier. In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIIIB metals, aluminum oxides, and mixtures thereof. Acidic support modifiers include those selected from the group consisting of TiO₂, ZrO₂, Nb₂O₅Ta₂O₅, Al₂O₃, B₂O₃, P₂O₅, and Sb₂O₃. Preferred acidic support modifiers include those selected from the group consisting of TiO₂, ZrO₂, Nb₂O₅, Ta₂O₅, and Al₂O₃. The acidic modifier may also include WO₃, MoO₃, Fe₂O₃, Cr₂O₃, V₂O₅, MnO₂, CuO, CO₂O₃, and Bi₂O₃.

In another embodiment, the support modifier may be a basic modifier that has a low volatility or no volatility. Such basic modifiers, for example, may be selected from the group consisting of: (i) alkaline earth oxides, (ii) alkali metal oxides, (iii) alkaline earth metal metasilicates, (iv) alkali metal metasilicates, (v) Group IIB metal oxides, (vi) Group IIB metal metasilicates, (vii) Group IIIB metal oxides, (viii) Group IIIB metal metasilicates, and mixtures thereof. In addition to oxides and metasilicates, other types of modifiers including nitrates, nitrites, acetates, and lactates may be used. Preferably, the support modifier is selected from the group consisting of oxides and metasilicates of any of sodium, potassium, magnesium, calcium, scandium, yttrium, and zinc, as well as mixtures of any of the foregoing. More preferably, the basic support modifier is a calcium silicate, and even more preferably calcium metasilicate (CaSiO₃). If the basic support modifier comprises calcium metasilicate, it is preferred that at least a portion of the calcium metasilicate is in crystalline form.

A preferred silica support material is SS61138 High Surface Area (HSA) Silica Catalyst Carrier from Saint Gobain N or Pro. The Saint-Gobain N or Pro SS61138 silica exhibits the following properties: contains approximately 95 wt. % high surface area silica; surface area of about 250 m²/g; a median pore diameter of about 12 nm; average pore volume of about 1.0 cm³/g as measured by mercury intrusion porosimetry and a packing density of about 0.352 g/cm³ (22 lb/ft³).

A preferred silica/alumina support material is KA-160 silica spheres from Sud Chemie having a nominal diameter of about 5 mm, a density of about 0.562 g/ml, an absorptivity of about 0.583 g H₂O/g support, a surface area of about 160 to 175 m²/g, and a pore volume of about 0.68 ml/g.

The catalyst compositions suitable for use with the present invention preferably are formed through metal impregnation of the modified support, although other processes such as chemical vapor deposition may also be employed. Such impregnation techniques are described in U.S. Pat. Nos. 7,608,744 and 7,863,489 and U.S. Pub. No. 2010/0029995 referred to above, the entireties of which are incorporated herein by reference.

In particular, the hydrogenation of acetic acid may achieve favorable conversion of acetic acid and favorable selectivity and productivity to ethanol. For purposes of the present invention, the term “conversion” refers to the amount of acetic acid in the feed that is converted to a compound other than acetic acid. Conversion is expressed as a mole percentage based on acetic acid in the feed. The conversion may be at least 10%, e.g., at least 20%, at least 40%, at least 50%, at least 60%, at least 70% or at least 80%. Although catalysts that have high conversions are desirable, such as at least 80% or at least 90%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid. It should be understood that each compound converted from acetic acid has an independent selectivity and that selectivity is independent from conversion. For example, if 60 mole % of the converted acetic acid is converted to ethanol, we refer to the ethanol selectivity as 60%. Preferably, the catalyst selectivity to ethoxylates is at least 60%, e.g., at least 70%, or at least 80%. As used herein, the term “ethoxylates” refers specifically to the compounds ethanol, acetaldehyde, and ethyl acetate. Preferably, the selectivity to ethanol is at least 80%, e.g., at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are not detectable. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram catalyst per hour, e.g., at least 400 grams of ethanol per kilogram catalyst per hour or at least 600 grams of ethanol per kilogram catalyst per hour, is preferred. In terms of ranges, the productivity preferably is from 100 to 3,000 grams of ethanol per kilogram catalyst per hour, e.g., from 400 to 2,500 per kilogram catalyst per hour or from 600 to 2,000 per kilogram catalyst per hour.

Operating under the conditions of the present invention may result in ethanol production on the order of at least 0.1 tons of ethanol per hour, e.g., at least 1 ton of ethanol per hour, at least 5 tons of ethanol per hour, or at least 10 tons of ethanol per hour. Larger scale industrial production of ethanol, depending on the scale, generally should be at least 1 ton of ethanol per hour, e.g., at least 15 tons of ethanol per hour or at least 30 tons of ethanol per hour. In terms of ranges, for large scale industrial production of ethanol, the process of the present invention may produce from 0.1 to 160 tons of ethanol per hour, e.g., from 15 to 160 tons of ethanol per hour or from 30 to 80 tons of ethanol per hour. Ethanol production from fermentation, due the economies of scale, typically does not permit the single facility ethanol production that may be achievable by employing embodiments of the present invention.

In various embodiments of the present invention, the crude ethanol product produced by the hydrogenation process, before any subsequent processing, such as purification and separation, will typically comprise acetic acid, ethanol and water. As used herein, the term “crude ethanol product” refers to any composition comprising from 5 to 70 wt. % ethanol and from 5 to 40 wt. % water. Exemplary compositional ranges for the crude ethanol product are provided in Table 1. The “Others” identified in Table 1 may include, for example, esters, ethers, aldehydes, ketones, alkanes, and carbon dioxide.

TABLE 1
CRUDE ETHANOL PRODUCT COMPOSITIONS

| | | Conc. | Conc. | |
|---|---|---|---|---|
| Component | Conc. (wt. %) | (wt. %) | (wt. %) | Conc. (wt. %) |
| Ethanol | 5 to 70 | 15 to 70 | 15 to 50 | 25 to 50 |
| Acetic Acid | 0 to 90 | 0 to 50 | 15 to 70 | 20 to 70 |
| Water | 5 to 40 | 5 to 30 | 10 to 30 | 10 to 26 |
| Ethyl Acetate | 0 to 30 | 0 to 20 | 1 to 12 | 3 to 10 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product comprises acetic acid in an amount less than 20 wt. %, e.g., less than 15 wt. %, less than 10 wt. % or less than 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

In one embodiment, the weight ratio of ethanol to water may be at least 0.18:1 or greater, e.g., at least 0.5:1 or at least 1:1. In terms of ranges the weight ratio of ethanol to water may be from 0.18:1 to 5:1, e.g., from 0.5:1 to 3:1 or from 1:1 to 2:1. Preferably the crude ethanol product has more ethanol than water compared to conventional fermentation processes of ethanol. In one embodiment, the lower amounts of water may require less energy to separate the ethanol and improves the overall efficiency of the process. Thus, in preferred embodiments, the amount of ethanol in the crude ethanol product is from 15 wt. % to 70 wt. %, e.g., from 20 wt. % to 70 wt. % or from 25 wt. % to 70 wt. %. Greater ethanol weight percents are particularly preferred.

### Ethanol Production System

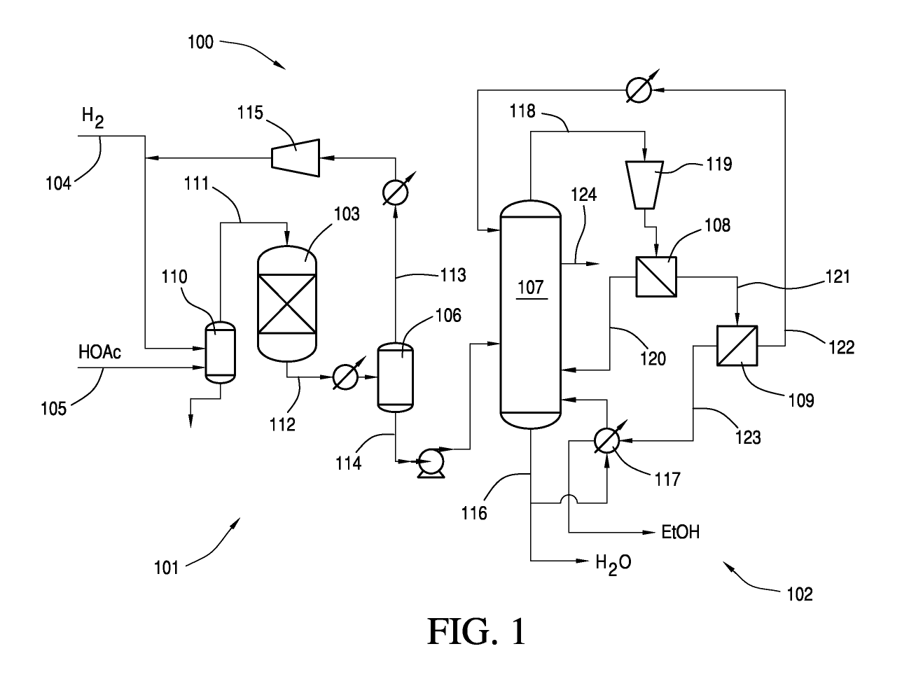

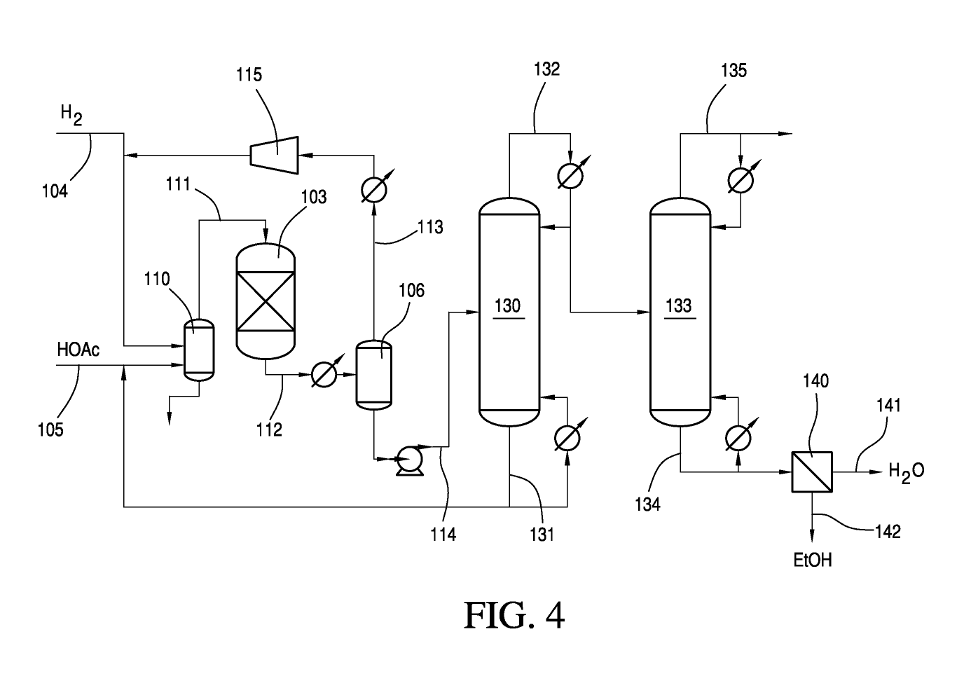

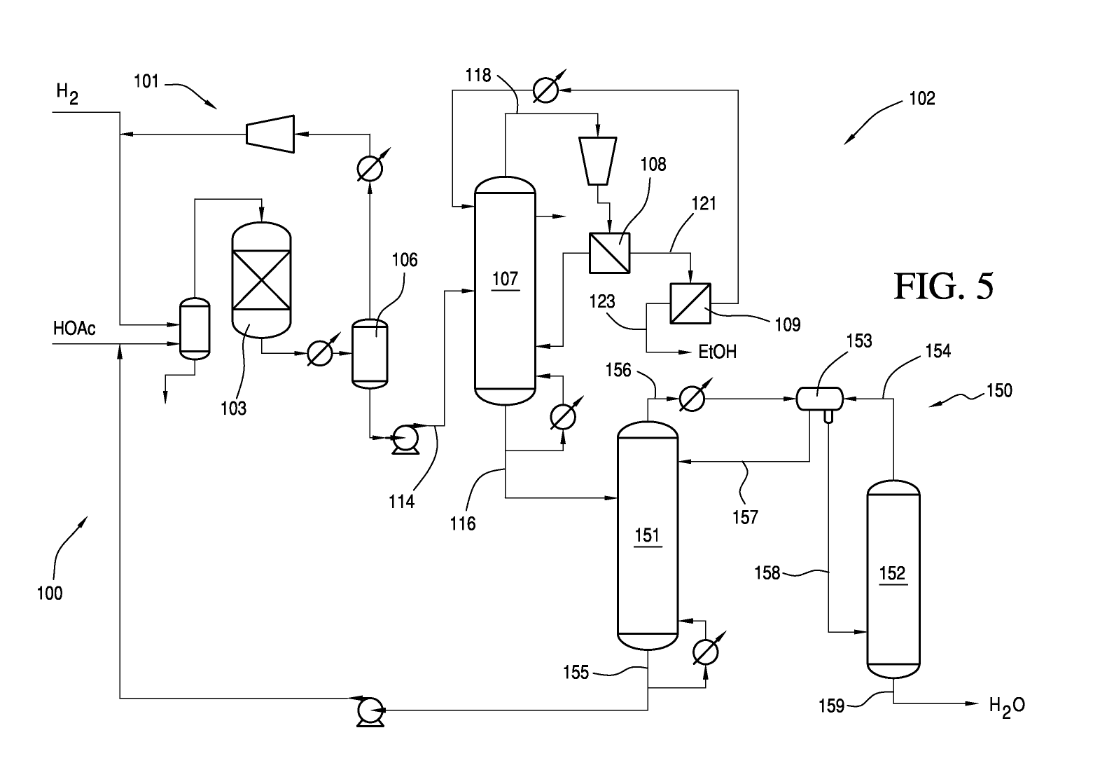

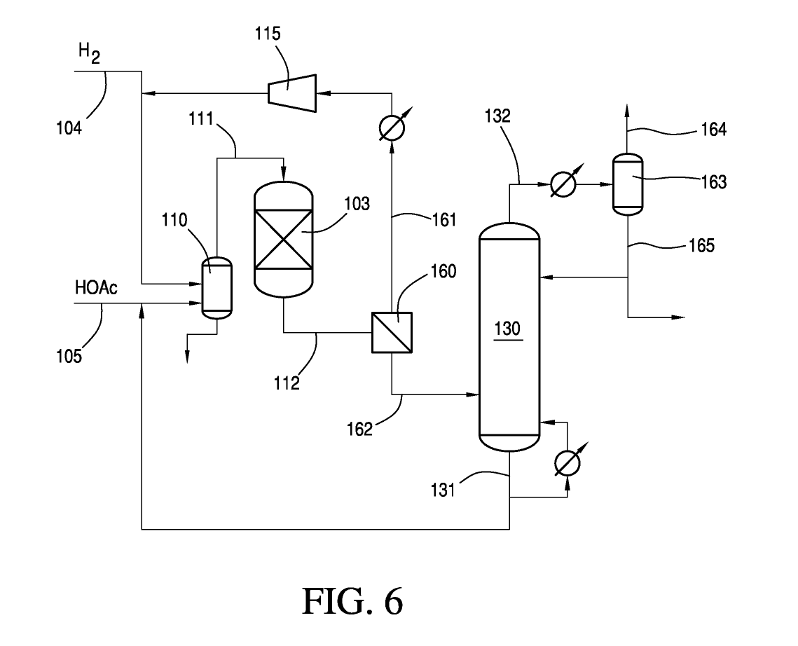

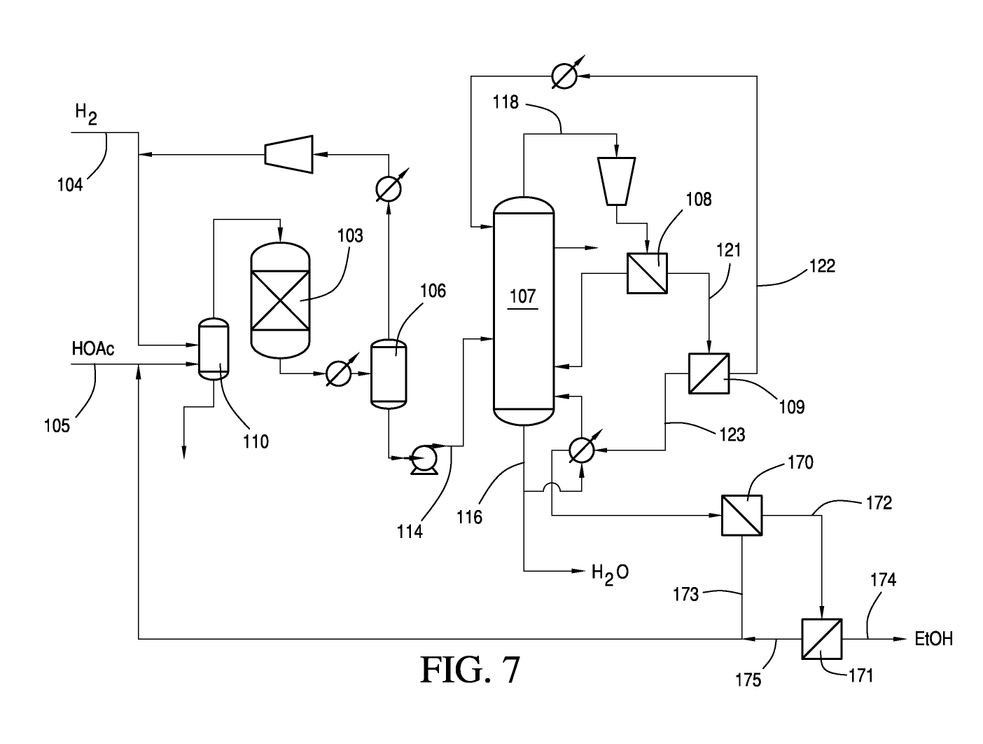

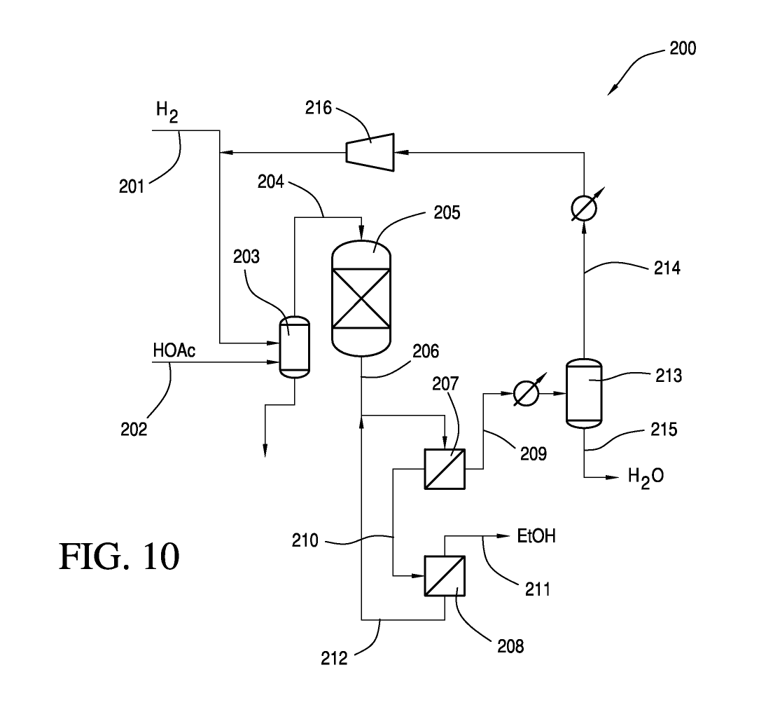

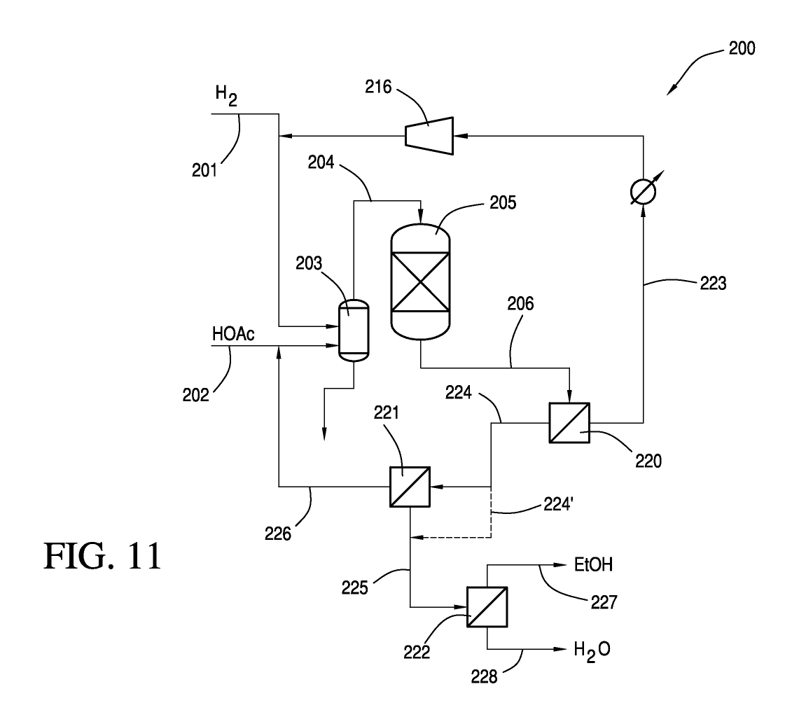

Various ethanol production systems are shown in FIGS. 1-11. In addition, the ethanol production systems, the system also includes separation columns and/or membranes. For example, FIGS. 1-3B use a combination of water permeable membranes with distillation column(s); FIGS. 4 and 5 use a combination of organic permeable membranes with distillation columns; FIG. 6 uses a hydrogen permeable membrane with a distillation column; FIGS. 7-9 use a combination of water permeable membranes, organic permeable membranes, and distillation columns. FIGS. 10 and 11 use a combination of membranes without distillation columns. These embodiments are exemplary and various membranes in each embodiment may be combined. For example, the membrane 160 in FIG. 6 may be used in place of or in combination with the separator (flasher) 106 shown in some of the other figures.

In hydrogenation system 100, hydrogen and acetic acid are fed to a vaporizer 110 via lines 104 and 105, respectively, to create a vapor feed stream in line 111 that is directed to reactor 103. In one embodiment, lines 104 and 105 may be combined and jointly fed to the vaporizer 110, e.g., in one stream containing both hydrogen and acetic acid. The temperature of the vapor feed stream in line 111 is preferably from 100° C. to 350° C., e.g., from 120° C. to 310° C. or from 150° C. to 300° C. Any feed that is not vaporized is removed from vaporizer 110, as shown, and may be recycled or discarded. In addition, although line 111 is shown as being directed to the top of reactor 103, line 111 may be directed to the side, upper portion, or bottom of reactor 103.

Reactor 103 contains the catalyst that is used in the hydrogenation of the carboxylic acid, preferably acetic acid. In one embodiment, one or more guard beds (not shown) may be used to protect the catalyst from poisons or undesirable impurities contained in the feed or return/recycle streams. Such guard beds may be employed in the vapor or liquid streams. Suitable guard bed materials are known in the art and include, for example, carbon, silica, alumina, ceramic, or resins. In one aspect, the guard bed media is functionalized to trap particular species such as sulfur or halogens. During the hydrogenation process, a crude ethanol product stream is withdrawn, preferably continuously, from reactor 103 via line 112.

The crude ethanol product stream in line 112 may be condensed and fed to flasher 106, which, in turn, separates the crude ethanol product 112 into a vapor stream 113 and a liquid stream 114. The flasher 106 preferably operates at a temperature of from 50° C. to 500° C., e.g., from 70° C. to 400° C. or from 100° C. to 350° C. In one embodiment, the pressure of flasher 106 preferably is from 50 kPa to 2000 kPa, e.g., from 75 kPa to 1500 kPa, or from 100 kPa to 1000 kPa. In one preferred embodiment, the temperature and pressure of the flasher is similar to the temperature and pressure of the reactor 103.

The vapor stream 113 exiting the flasher 106 may comprise hydrogen and hydrocarbons, which may be purged and/or returned to reaction zone 101 via line 113. As shown in FIG. 1, the returned portion of the vapor stream 113 passes through compressor 115 and is combined with the hydrogen feed and co-fed to vaporizer 110.

The liquid stream 114 from flasher 106 is withdrawn and pumped into distillation column 107. In one embodiment, the contents of liquid stream 114 are substantially similar to the crude ethanol product obtained from the reactor, except that the composition has been depleted of hydrogen, carbon dioxide, methane and/or ethane, which are removed by the flasher 106. Accordingly, liquid stream 114 may also be referred to as a crude ethanol product. Exemplary compositions of liquid stream 114 are provided in Table 2. It should be understood that liquid stream 114 may contain other components, not listed.

TABLE 2
LIQUID STREAM 114

| | Conc. (wt. %) | Conc. (wt. %) | Conc. (wt. %) |
|---|---|---|---|
| Ethanol | 5 to 70 | 10 to 60 | 15 to 50 |
| Acetic Acid | <90 | 5 to 80 | 15 to 70 |
| Water | 5 to 35 | 5 to 30 | 10 to 30 |
| Ethyl Acetate | <20 | 0.001 to 15 | 1 to 12 |
| Acetaldehyde | <10 | 0.001 to 3 | 0.1 to 3 |
| Acetal | <5 | 0.001 to 2 | 0.005 to 1 |
| Acetone | <5 | 0.0005 to 0.05 | 0.001 to 0.03 |
| Other Esters | <5 | <0.005 | <0.001 |
| Other Ethers | <5 | <0.005 | <0.001 |
| Other Alcohols | <5 | <0.005 | <0.001 |

The amounts indicated as less than (<) in the tables throughout present application are preferably not present and if present may be present in trace amounts or in amounts greater than 0.0001 wt. %.

The “other esters” in Table 2 may include, but are not limited to, ethyl propionate, methyl acetate, isopropyl acetate, n-propyl acetate, n-butyl acetate or mixtures thereof. The “other ethers” in Table 2 may include, but are not limited to, diethyl ether, methyl ethyl ether, isobutyl ethyl ether or mixtures thereof. The “other alcohols” in Table 2 may include, but are not limited to, methanol, isopropanol, n-propanol, n-butanol or mixtures thereof. In one embodiment, the liquid stream 114 may comprise propanol, e.g., isopropanol and/or n-propanol, in an amount from 0.001 to 0.1 wt. %, from 0.001 to 0.05 wt. % or from 0.001 to 0.03 wt. %. In should be understood that these other components may be carried through in any of the distillate or residue streams described herein and will not be further described herein, unless indicated otherwise.

In preferred embodiments, the crude ethanol product or liquid stream 114 fed to distillation column 107 comprises acetic acid in an amount of less than 20 wt. %, e.g., of less than 15 wt. %, less than 10 wt. % or less than 5 wt. %. In embodiments having lower amounts of acid acetic, the conversion of acetic acid in the reactor 103 is preferably greater than 75%, e.g., greater than 85% or greater than 90%. In addition, the selectivity to ethanol is preferably high, and is greater than 75%, e.g., greater than 85% or greater than 90%.

The distillation columns used in combination with membranes may comprise any distillation column capable of separation and/or purification. Each column preferably comprises a tray column having from 1 to 150 trays, e.g., from 10 to 100, from 20 to 95 trays or from 30 to 75 trays. The trays may be sieve trays, fixed valve trays, movable valve trays, or any other suitable design known in the art. In other embodiments, a packed column may be used. For packed columns, structured packing or random packing may be employed. The trays or packing may be arranged in one continuous column or they may be arranged in two or more columns such that the vapor from the first section enters the second section while the liquid from the second section enters the first section, etc.

For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

The temperatures and pressures employed in the columns may vary. As a practical matter, pressures from 10 kPa to 3000 kPa will generally be employed in these zones although in some embodiments subatmospheric pressures or superatmospheric pressures may be employed. Temperatures within the various zones will normally range between the boiling points of the composition removed as the distillate and the composition removed as the residue. As will be recognized by those skilled in the art, the temperature at a given location in an operating distillation column is dependent on the composition of the material at that location and the pressure of column. In addition, feed rates may vary depending on the size of the production process and, if described, may be generically referred to in terms of feed weight ratios.

The associated condensers and liquid separation vessels that may be employed with each of the distillation columns may be of any conventional design and are simplified in the figures. Heat may be supplied to the base of each column or to a circulating bottom stream through a heat exchanger or reboiler. Other types of reboilers, such as internal reboilers, may also be used in some embodiments. The heat that is provided to reboilers may be derived from any heat generated during the process that is integrated with the reboilers or from an external source such as another heat generating chemical process or a boiler. Although one reactor and flasher are shown, additional reactors, flashers, condensers, heating elements, and other components may be used in embodiments of the present invention. As will be recognized by those skilled in the art, various condensers, pumps, compressors, reboilers, drums, valves, connectors, separation vessels, etc., normally employed in carrying out chemical processes may also be combined and employed in the processes of the present invention.

### Water Permeable Membranes

Suitable water permeable membranes include hydrophilic polymer membranes, such as crosslinked polyvinyl alcohol membranes, polyethylene glycol membranes, polyethersulfone membranes, and perfluoropolymer membranes. When separating a crude ethanol product, water is separated as the permeate stream and other components in the crude product are separated as the retentate stream. For purposes of the present invention hydrophobic polymer membranes that retain water may also be used.

In the embodiment shown in FIG. 1, liquid stream 114 is introduced in the middle part of an ethanol product column 107, e.g., second quarter or third quarter. In one embodiment, the distillation column 107 may be a dephlegmator column. In column 107, water, acetic acid, and other heavy components, if present, are removed from the liquid stream 114 and are withdrawn, preferably continuously, as residue in line 116. Residue 116 is preferably purged from the system 100 via line 116. A portion of the residue in line 116 may be directed to a reboiler 117 for supplying heat to the column 107. In one embodiment, the residue comprises water in an amount of at least 60 wt. %, e.g., at least 80 wt. % or at least 90 wt. %. Residue in line 116 may also comprise any other heavy components, such as acetic acid.

Column 107 also forms a distillate stream 118, as a vapor stream. A side stream from column 107 comprising fusel oils may also be withdrawn via line 124. When column 107 is operated under standard atmospheric pressure, the temperature of the residue exiting in line 116 from column 107 preferably is from 70° C. to 115° C., e.g., from 80° C. to 110° C. or from 85° C. to 105° C. The temperature of the distillate exiting in line 118 from column 107 preferably is from 60° C. to 110° C., e.g., from 70° C. to 100° C. or from 75° C. to 95° C. In other embodiments, the pressure of column 107 may range from 0.1 kPa to 510 kPa, e.g., from 1 kPa to 475 kPa or from 1 kPa to 375 kPa. The distillate stream 118 passes through a compressor 119 and is fed to a water permeable membrane 108. Compressor 119 supplies a driving force for a portion of the distillate stream 118 to pass through the water permeable membrane 108. The water permeable membrane 108 has a selectivity for water and separates a water stream 120 (permeate) and an initial ethanol stream 121 (retentate) that comprises ethanol and minor portions of water. In one embodiment, the water stream 120 comprises water in an amount of at least 60 wt. %, e.g., at least 80 wt. % or at least 90 wt. %. In one embodiment, the initial ethanol stream 121 comprises ethanol in an amount of at least 60 wt. %, e.g., at least 70 wt. % or at least 85 wt. % and water in an amount of less than 40 wt. %, e.g., less than 30 wt. % or less than 15 wt. %. Water permeable membrane 108 preferably reduces the water concentration of initial ethanol stream 121 by at least 60% based on the water concentration in the distillate stream 118, e.g., at least 80 or at least 90%. Water permeable membrane 108 may comprise a hydrophilic polymer membrane, such as a crosslinked polyvinyl alcohol membrane.

In an embodiment, water stream 120 may be returned to column 107. A portion of water stream 120 may be fed to column 107 such that the composition of water stream 120 is substantially similar to the composition of the liquid on the tray(s) in that portion of the column 107.

In some embodiments, initial ethanol stream 121 may be withdrawn as an ethanol product. The ethanol product obtained from the initial ethanol stream 121 may be suitable for industrial grade ethanol. However, in some embodiments, for example in order to obtain fuel grade or anhydrous ethanol, it may be preferred to remove the remaining water in the initial ethanol stream 121. As shown in FIG. 1, initial ethanol stream 121 is fed to second water permeable membrane 109. Preferably, the initial ethanol stream 121 comprises a lower water concentration and a higher ethanol concentration than distillate stream 118. In some optional embodiments, an additional compressor (not shown) may be used to compress the initial ethanol stream 121. The second water permeable membrane 109 removes the remaining water from the initial ethanol stream 121 as the second water stream 122 (permeate) and forming a dehydrated ethanol stream 123 (retentate) that comprises or consists essentially of ethanol. Second water permeable membrane 109 may comprise a crosslinked polyvinyl alcohol membrane. The second water stream 122 may be condensed and refluxed to the upper portion of column 107. The second water stream 122 comprises substantially all of the water from the initial ethanol stream 121 and a small amount of ethanol. In one embodiment, the composition of second water stream 122 contains less water than liquid feed stream 114. This may allow for more efficient separation of the ethanol and water in column 107. In one embodiment, the second water stream 122 comprises ethanol in an amount of at least 25 wt. %, e.g., at least 30 wt. % or at least 40 wt. % and water in an amount of less than 75 wt. %, e.g., less than 70 wt. % or less than 60 wt. %.

In preferred embodiments, the heat energy of the dehydrated ethanol stream 123 may be used to supply a portion of the heat for reboiler 117. The heat energy of the dehydrated ethanol stream 123 may also be integrated to supply heat to other portions of the system 100.

The dehydrated ethanol stream 123 preferably comprises ethanol in an amount greater than 85 wt. %., e.g. greater than 92 wt. %, greater than 95 wt. % or greater than 99 wt. %. The dehydrated ethanol stream 123 may be condensed to recover a finished ethanol product. In some embodiments, the dehydrated ethanol stream 123 may be further processed in one or more distillation column and/or adsorption beds. This processing may be advantageous when the dehydrated ethanol stream 123 contains other compounds, such as ethyl acetate and/or acetaldehyde.

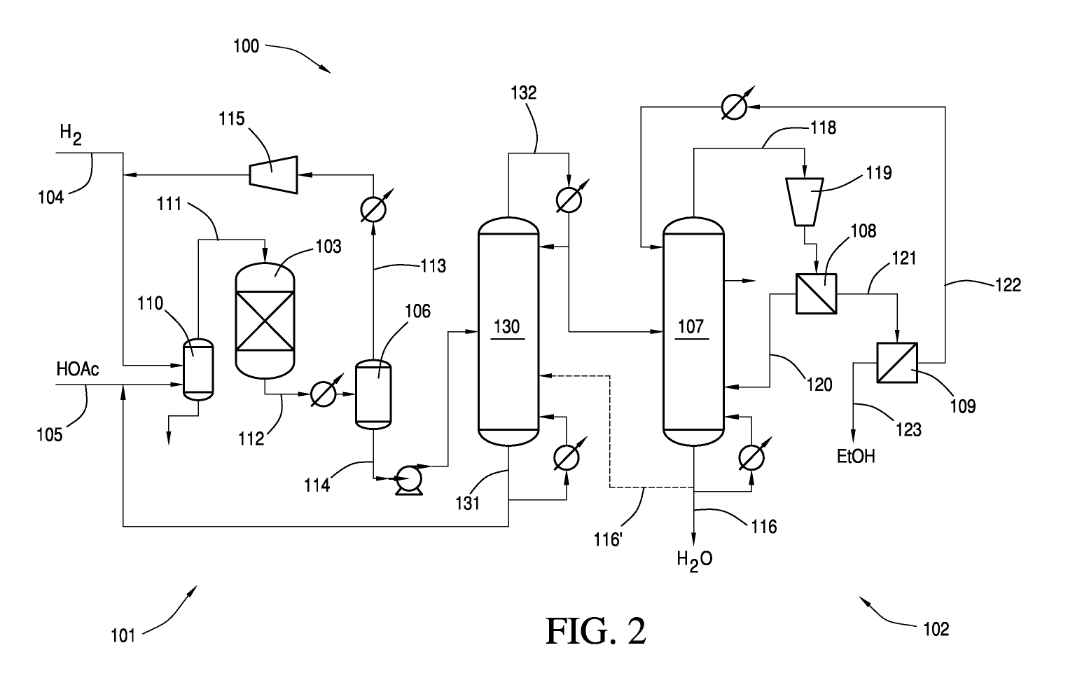

In FIG. 2, there is provided an additional column 130, also referred to as an “acid separation column.” Liquid stream 114 is introduced in the middle part of column 130, e.g., second quarter or the third quarter. In addition, in some embodiments, a portion of the residue from the ethanol product column 107 may be directed to column 130 via line 116′. In acid separation column 130, acetic acid, a portion of the water, and other heavy components, if present, are removed from the composition in line 114 and are withdrawn, preferably continuously, as residue 131. Some or all of the residue may be directly or indirectly returned and/or recycled back to reaction zone 101 via line 131. Reducing the amount of heavies to be purged may improve efficiencies of the process while reducing byproducts. Column 130 also fauns an overhead distillate, which is withdrawn in line 132, and which may be condensed and refluxed, for example, at a ratio of from 10:1 to 1:10, e.g., from 3:1 to 1:3 or from 1:2 to 2:1. The distillate in line 132 preferably comprises ethanol, ethyl acetate, and water, along with other impurities. Distillate 132 is preferably introduced to column 107, and ethanol is separated using water permeable membranes 108 and 109 as discussed above in FIG. 1.

When column 130 is operated under about 170 kPa, the temperature of the residue exiting in line 131 preferably is from 90° C. to 130° C., e.g., from 95° C. to 120° C. or from 100° C. to 115° C. The temperature of the distillate exiting in line 132 from column 130 preferably is from 60° C. to 90° C., e.g., from 65° C. to 85° C. or from 70° C. to 80° C. In other embodiments, the pressure of column 130 may range from 0.1 kPa to 510 kPa, e.g., from 1 kPa to 475 kPa or from 1 kPa to 375 kPa. Exemplary components of the distillate and residue compositions for column 130 are provided in Table 3 below. It should be understood that the distillate and residue may also contain other components, not listed.

TABLE 3
ACID SEPARATION COLUMN 130

| | Conc. (wt. %) | Conc. (wt. %) | Conc. (wt. %) |
|---|---|---|---|
| Distillate | | | |
| Ethanol | 20 to 75 | 30 to 70 | 40 to 65 |
| Water | 10 to 40 | 15 to 35 | 20 to 35 |
| Acetic Acid | <2 | 0.001 to 0.5 | 0.01 to 0.2 |
| Ethyl Acetate | <60 | 5.0 to 40 | 10 to 30 |
| Acetaldehyde | <10 | 0.001 to 5 | 0.01 to 4 |
| Acetal | <0.1 | <0.1 | <0.05 |
| Acetone | <0.05 | 0.001 to 0.03 | 0.01 to 0.025 |
| Residue | | | |
| Acetic Acid | 60 to 100 | 70 to 95 | 85 to 92 |
| Water | <30 | 1 to 20 | 1 to 15 |
| Ethanol | <1 | <0.9 | <0.07 |

Some species, such as acetals, may decompose in column 107 such that very low amounts, or even no detectable amounts, of acetals remain in the distillate or residue. In addition, a non-catalyzed equilibrium reaction between acetic acid and ethanol or between ethyl acetate and water may occur in the crude ethanol product after it exits reactor 103. Depending on the concentration of acetic acid in the crude ethanol product, this equilibrium may be driven toward formation of ethyl acetate. This equilibrium may be regulated using the residence time and/or temperature of crude ethanol product.

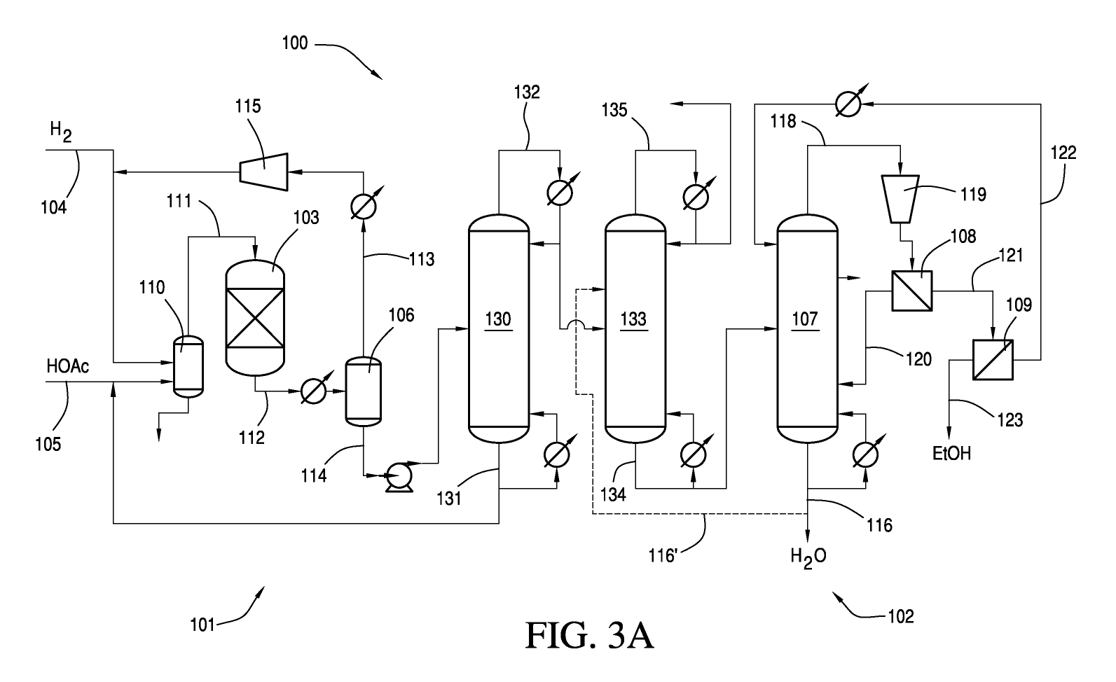

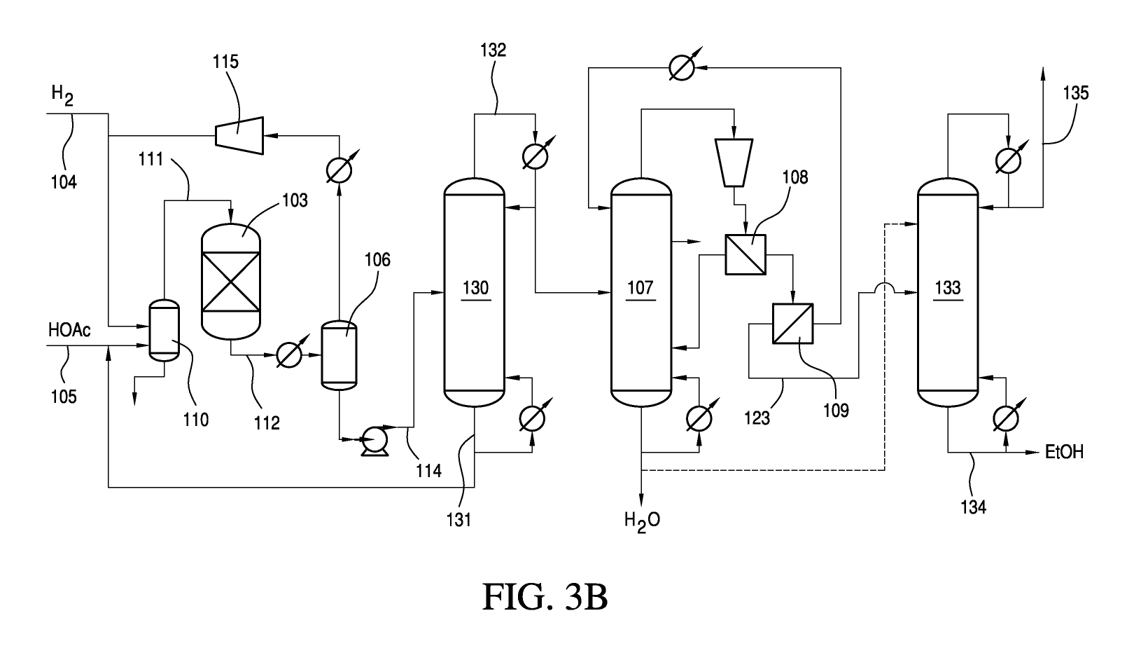

FIGS. 3A and 3B are similar to FIG. 1, but includes two additional columns, 130 and 133. Acid separation column 130 is described above in FIG. 2. In FIG. 3A, distillate in line 132 is introduced to column 133, also referred to as a “light ends column,” preferably in the top portion of column 133, e.g., top third. As one example, when a column having 25 trays is used without water extraction, line 132 may be introduced at tray 17. A column having 30 trays is used without water extraction, line 132 may be introduced at tray 2. In one embodiment, the column 133 may be an extractive distillation column. In such embodiments, an extraction agent, such as, for example water, may be added to column 133. If the extraction agent comprises water, it may be obtained from an external source or from an internal return/recycle line from one or more of the other columns, as shown by line 116′ from the residue of column 107.

In some embodiments, the light ends column 133 may be an extractive distillation column. Suitable extractive agents may include, for example, dimethylsulfoxide, glycerine, diethylene glycol, 1-naphthol, hydroquinone, N,N′-dimethylformamide, 1,4-butanediol; ethylene glycol-1,5-pentanediol; propylene glycol-tetraethylene glycol-polyethylene glycol; glycerine-propylene glycol-tetraethylene glycol-1,4-butanediol, ethyl ether, methyl formate, cyclohexane, N,N′-dimethyl-1,3-propanediamine, N,N′-dimethylethylenediamine, diethylene triamine, hexamethylene diamine and 1,3-diaminopentane, an alkylated thiopene, dodecane, tridecane, tetradecane, chlorinated paraffins, or a combination thereof.

Light ends column 133 may be a tray column or packed column. In one embodiment, column 133 is a tray column having from 5 to 70 trays, e.g., from 15 to 50 trays or from 20 to 45 trays. Although the temperature and pressure of column 133 may vary, when at about 20 kPa to 70 kPa, the temperature of the residue exiting in line 134 from column 133 preferably is from 30° C. to 75° C., e.g., from 35° C. to 70° C. or from 40° C. to 65° C. The temperature of the distillate exiting in line 135 from column 133 preferably is from 20° C. to 55° C., e.g., from 25° C. to 50° C. or from 30° C. to 45° C. Light ends column 133 may operate at a reduced pressure, near or at vacuum conditions, to further favor separation of ethyl acetate and ethanol. In other embodiments, the pressure of column 133 may range from 0.1 kPa to 510 kPa, e.g., from 1 kPa to 475 kPa or from 1 kPa to 375 kPa. Exemplary components of the distillate and residue compositions for column 133 are provided in Table 4 below. It should be understood that the distillate and residue may also contain other components, not listed.

TABLE 4
LIGHT ENDS COLUMN 133

| | Conc. (wt. %) | Conc. (wt. %) | Conc. (wt. %) |
|---|---|---|---|
| Distillate | | | |
| Ethyl Acetate | 10 to 90 | 25 to 90 | 50 to 90 |
| Acetaldehyde | 1 to 25 | 1 to 15 | 1 to 8 |
| Water | 1 to 25 | 1 to 20 | 4 to 16 |
| Ethanol | <30 | 0.001 to 15 | 0.01 to 5 |
| Acetal | <5 | 0.001 to 2 | 0.01 to 1 |
| Residue | | | |
| Water | 30 to 70 | 30 to 60 | 30 to 50 |
| Ethanol | 20 to 75 | 30 to 70 | 40 to 70 |
| Ethyl Acetate | <3 | 0.001 to 2 | 0.001 to 0.5 |
| Acetic Acid | <0.5 | 0.001 to 0.3 | 0.001 to 0.2 |

The weight ratio of ethanol in the residue to distillate of column 133 preferably is at least 2:1, e.g., at least 6:1, at least 8:1, at least 10:1 or at least 15:1. The weight ratio of ethyl acetate in the residue to distillate preferably is less than 0.4:1, e.g., less than 0.2:1 or less than 0.1:1. In embodiments that use an extractive column with water as an extraction agent in the column 133, the weight ratio of ethyl acetate in the residue to ethyl acetate in the distillate of column 133 is less than 0.1:1.

As shown in FIG. 3A, the residue from the bottom of column 133, which comprises ethanol and water, is fed via line 134 to column 107. Ethanol is separated using membranes 108 and 109 as discussed above in FIG. 1. The distillate in line 135 preferably is refluxed as shown in FIG. 3A, for example, at a reflux ratio of from 1:10 to 10:1, e.g., from 1:5 to 5:1 or from 1:3 to 3:1. The distillate from column 133 may be purged and fed to an esters process or removed as an ethyl acetate solvent. Alternatively, since distillate from column 133 contains ethyl acetate, all or a portion of the distillate from column 133 may be recycled to reaction zone 101 via line 135 in order to convert the ethyl acetate to additional ethanol. All or a portion of distillate 135 may be recycled to reactor 103, and may be co-fed with the acetic acid feed line 105. In another embodiment, the distillate in line 135 may be further purified to remove impurities, such as acetaldehyde, using one or more additional columns.

In FIG. 3B, distillate in line 132 is introduced to ethanol product column 107. Ethanol is separated using water permeable membranes 108 and 109 as discussed above in FIG. 1. A portion or all of the dehydrated ethanol stream 123, and optionally the ethanol product stream 108, may be introduced to column 133. In contrast to FIG. 3A, the dehydrated ethanol stream 123 contains less water than distillate 132. Light ends column 133 may be used to remove the ethyl acetate and acetaldehydes that pass along with the ethanol in the dehydrated ethanol stream 123. These compounds are separated and removed in the distillate of column 133 in line 135. The residue of column 133 in line 134 comprises an ethanol product.

FIG. 4 is similar to FIG. 3A, but replaces ethanol product column 107 and the associated membranes 108 and 109, with water permeable membrane 140. Water permeable membrane 140 may comprise one or more membranes arranged in an array. In FIG. 4, residue in line 134 from light ends column 133, which comprises ethanol and water, is fed to water permeable membrane 140. Water permeable membrane 140 is selective for water and separates a water stream 141 (permeate) and an ethanol product stream 142 and minor portions of water (retentate). In one embodiment, the water stream 141 comprises water in an amount of at least 60 wt. %, e.g., at least 80 wt. % or at least 90 wt. %. In one embodiment, the ethanol product stream 142 comprises ethanol in an amount of at least 60 wt. %, e.g., at least 70 wt. % or at least 85 wt. % and water in an amount of less than 40 wt. %, e.g., less than 30 wt. % or less than 15 wt. %. Water permeable membrane 140 preferably reduces the water concentration of the residue 134 of column 133 by at least 60% based on the water concentration in the residue stream 134, e.g., at least 80% or at least 90%. Additional membranes may be used in parallel or in series with water permeable membrane 140 to achieve the desirable water concentration in ethanol product stream 142.

### Acid Treatment

FIG. 5 shows a separation system 100 similar to FIG. 1 having ethanol product column 107 in separation zone 102, and further comprising a weak acid recovery zone 150. Weak acid recovery zone 150 may be added to any of the separation systems used throughout the present invention to recover acid from any acid stream. In one embodiment, weak acid recovery zone 150 comprises an azeotropic acid-water separator column 151, effluent still 152, and decanter 153. In some embodiments, an extractor (not shown) may also be provided to initially treat the residue 116 before it is fed to separator column 151. In those embodiments, an extractor may be used when the concentration of acetic acid in residue 116 is less than 50 wt. %.

FIG. 5 illustrates a process for purifying the residue in 116 of ethanol product column 107. As shown in FIG. 5, residue 116, which comprises acetic acid and water, is preferably fed to separator column 151. In one embodiment, the residue 116 may comprise a dilute acid stream, comprising water and acetic acid. Generally it is difficult to separate mixtures of acetic acid and water, even though acetic acid does not form an azeotrope with water. In one embodiment, separator column 151 may comprise an extraction agent, such as a compound capable of forming an azeotrope with water, but which preferably does not form an azeotrope with acetic acid. Suitable azeotroping compounds include ethyl acetate, propyl acetate, isopropyl acetate, butyl acetate, vinyl acetate, diisopropyl ether, carbon disulfide, tetrahydrofuran, isopropanol, ethanol, and C₃-C₁₂ alkanes. Ethyl acetate, isopropyl acetate and diisopropyl ether are preferred azeotrope compounds. Separator column 151 produces a distillate in line 156, which comprises water and the extraction agent, such as ethyl acetate, and a residue in line 155 comprising acetic acid. Preferably, the residue 155 comprises acetic acid that contains little or no water (dry acetic acid). In one embodiment, the amount of water in residue 155 is less than 3 wt. %, e.g., less than 1 wt. % or less than 0.5 wt. %. Residue 155 may be directly or indirectly introduced to reaction zone 101 by adding residue 155 with the acetic acid feed 105 to vaporizer 110. The distillate 156 is condensed overhead and is biphasically separated in a decanter 153 into a light phase in line 157 that comprises the azeotroping compound, such as ethyl acetate, and a heavy phase in line 158 that comprises water. The light phase in line 157 may be refluxed to separator column 151 as shown in FIG. 5. Heavy phase 158 is fed to effluent still 152 to recover an effluent stream comprising water in line 159 and a vapor stream of the azeotrope compound, i.e., ethyl acetate, in line 154. Vapor stream 154 may be directly or indirectly fed to the decanter 153. Water stream 159 may be purged from the system.

Depending on the water and acetic acid concentrations in residue ethanol product column 107 and the flow rate of that stream, line 116 may be treated in one or more of the following other processes. Depending on the composition, the residue stream may be: (i) entirely or partially recycled to the hydrogenation reactor, (ii) separated into acid and water streams, (iii) neutralized, (iv) reacted with an alcohol to consume the unreacted acetic acid, or (v) disposed to a waste water treatment facility.

When neutralizing the acetic acid, it is preferred that the residue in line 116 comprises less than 10 wt. % acetic acid. Acetic acid may be neutralized with any suitable alkali or alkaline earth metal base, such as sodium hydroxide or potassium hydroxide. When reacting acetic acid with an alcohol, it is preferred that the residue comprises less than 50 wt. % acetic acid. The alcohol may be any suitable alcohol, such as methanol, ethanol, propanol, butanol, or mixtures thereof. The reaction forms an ester that may be integrated with other systems, such as carbonylation production or an ester production process. Preferably, the alcohol comprises ethanol and the resulting ester comprises ethyl acetate. Optionally, the resulting ester may be fed to the hydrogenation reactor.

In some embodiments, when the residue comprises very minor amounts of acetic acid, e.g., less than 5 wt. % or less than 0.5 wt. %, the residue may be disposed of to a waste water treatment facility without further processing. The organic content, e.g., acetic acid content, of the residue beneficially may be suitable to feed microorganisms used in a waste water treatment facility.

### Hydrogen Permeable Membrane Embodiment

Hydrogen permeable membranes are suitable for the separation of vapor phase separation of a crude ethanol product. In one embodiment, the hydrogen permeable membrane is a polymer based membrane that operates at a maximum temperature of 100° C. and at a pressure of greater than 500 kPa, e.g., greater than 700 kPa. In another embodiment, the hydrogen permeable membrane is a palladium-based membrane, such as palladium-based alloy with copper, yttrium, ruthenium, indium, lead, and/or rare earth metals, that has a high selectivity for hydrogen. Suitable palladium-based membranes are described in Burkhanov, et al., “Palladium-Based Alloy Membranes for Separation of High Purity Hydrogen from Hydrogen-Containing Gas Mixtures,” Platinum Metals Rev., 2011, 55, (1), 3-12, the entirety of which is incorporated by reference. Efficient hydrogen separation palladium-based membranes generally have high hydrogen permeability, low expansion when saturated with hydrogen, good corrosion resistance and high plasticity and strength during operation at temperatures of from 100° C. to 900° C., e.g. from 300° C. to 700° C. Because the crude ethanol product may contain unreacted acid, hydrogen permeable membrane should tolerate acidic conditions of about pH 3 to 4.

FIG. 6 shows a column separation scheme in which the flasher is replaced by a membrane 160. A crude ethanol product stream is withdrawn, preferably continuously, from reactor 103 via line 112 and is fed to membrane 160. The driving force for crude ethanol product stream 112 is preferably provided by reactor 103 and optionally one or more compressors (not shown). Hydrogen permeable membrane 160 has a high selectivity for hydrogen. Although, other gases, such as methane, ethane and/or carbon dioxide may also permeate through the membrane to some extent. This stream may be superheated and compressed before returning to the vaporizer. Retentate stream 162 may comprise ethanol, water, acetic acid, ethyl acetate, and other heavy components. The hydrogen stream 161 preferably comprises hydrogen in an amount greater than 85 wt. %., e.g. greater than 92 wt. %, greater than 95 wt. % or greater than 99 wt. %. Retentate stream 162 is in a vapor phase and is fed directly to column 130. The heat of the retentate stream 162 may be used to provide the necessary heat for column 130. In some embodiments, the reboiler of column 130 may be needed on start-up. Column 130 forms a residue in line 131 that comprises acetic acid and may be returned to reaction zone 101 as shown, purged or treated in a weak acid recovery.

In addition, the distillate of column 130 may be condensed and may be purified using schemes as described above. In some optional embodiments, there also may be provided a flasher 163. Flasher 163 operates at conditions sufficient to provide a vapor stream and a liquid stream. The vapor stream in line 164 exiting the flasher 163 may comprise hydrogen and hydrocarbons, which may be purged as shown and/or returned to reaction zone 101. The liquid 165 from flasher 163 is withdrawn and may be refluxed to column 130 and introduced to columns 107 or 133 as described above in FIGS. 2, 3A, 3B, and 4. Hydrogen permeable membrane may be used with the other separation schemes discussed herein to replace the flasher when it is desirable to feed the second distillation in vapor phase.

### Organic Permeable Membrane

Organic permeable membranes may include ethanol permeable membranes or ethyl acetate permeable membranes. Ethanol and ethyl acetate may be separated from one another using such membranes. The organic permeable membranes may separate a stream having both organics and aqueous and separate the organics in the permeate stream and the aqueous in the retentate stream. An ethanol permeable membrane may be used to separate ethanol and ethyl acetate into a permeate stream of ethanol and a retentate stream of ethyl acetate. Suitable organic permeable membranes include polycrystalline silicalite membranes, PDMS membranes, and NaY type zeolite membranes.

FIG. 7 shows a similar ethanol product column 107 and membranes 108 and 109 as shown in FIG. 1, and also includes ethanol permeable membranes 170 and 171. An acid separation column 130 as shown in FIG. 2 may also be used with the organic permeable membranes. Dehydrated ethanol stream 123, the retentate of membrane 109, is directed to one or more organic permeable membranes 170 and 171. For certain types of ethanol, it is desirable to remove ethyl acetate, which may form in reactor 103 and/or ethanol product column 107.

Dehydrated ethanol product stream 123 from water permeable membrane 109 comprises ethanol and ethyl acetate, and minor amounts of water as discussed above. The ethanol permeable membrane 170 has a selectivity for ethanol and generates an ethanol product stream 172 (permeate) and an ethyl acetate stream 173 (retentate). In one embodiment, the ethanol product stream 172 comprises a higher concentration of ethanol than the dehydrated ethanol product stream 123. The ethanol product stream 172 may be fed through another ethanol permeable membrane 171, which also has a higher selectivity for ethanol, to further remove any undesirable materials from ethanol product stream 172. In some embodiments, ethanol product stream 172 may be withdrawn as an ethanol product. The ethanol permeable membrane 171 separates the ethanol product stream 172 into a final ethanol product stream 174 (permeate) and a second ethyl acetate stream 175 (retentate). In one embodiment, the final ethanol product stream 174 comprises ethanol in an amount of at least 90 wt. %, e.g., at least 95 wt. % or at least 98 wt. %. In one embodiment, the second ethyl acetate stream 175 may be combined with ethyl acetate stream 173 and co-fed to the vaporizer, directly or indirectly, to generate more ethanol. Optionally, a portion of the streams may recycle back through the same membrane to obtain higher product purity. For example, a portion of the permeate stream 172 may be fed through the ethanol permeable membrane 170 to result in an ethanol permeate stream having a lesser amount of ethyl acetate than the permeate stream 172.

It should be understood that membranes with a selectivity for ethyl acetate may be used in place of ethanol permeable membranes 170 and 171. In such situations, the mixture of ethyl acetate and ethanol may be separated into retentate streams that comprise ethanol, and permeate streams that comprise ethyl acetate.

In optional embodiments, a portion of second ethyl acetate stream 175 may be introduced to an acetaldehyde column, as described below, to recover an acetaldehyde stream suitable for returning to reaction zone 101.

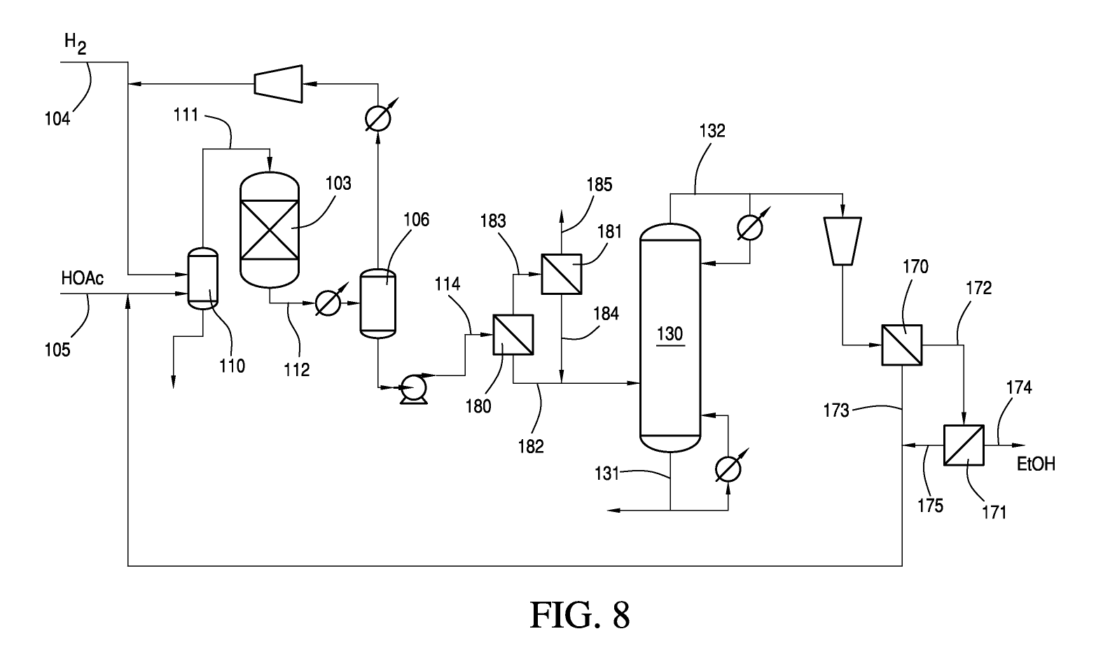

FIG. 8 illustrates another separation system having organic permeable membranes. In this embodiment, water is removed using water permeable membranes before acetic acid is removed from the crude ethanol product. As shown in FIG. 8, crude ethanol feed 114 is fed through a water permeable membrane 180. Water permeable membrane 180 has a selectivity for water and separates a water stream 183 (permeate) and a first retentate stream 182 that comprises ethanol, ethyl acetate and acetic acid. In one embodiment, the water stream 183 comprises water in an amount of at least 60 wt. %, e.g., at least 70 wt. % or at least 85 wt. %. In one embodiment, the first retentate stream 182 comprises ethanol in an amount of at least 50 wt. %, e.g., at least 60 wt. % or at least 75 wt. %. Water permeable membrane 180 may comprise a hydrophilic polymer membrane such as a crosslinked polyvinyl alcohol membrane.

It should be noted that one or more membranes may be used in series or in parallel in order to achieve the desirable purity of the final ethanol product. In addition, it should be noted that either the permeate and/or the retentate stream may pass through additional membranes. Also a stream may be recycle through the same membrane to remove undesirable materials. For example, if it is desirable to obtain crude ethanol product with reduced amount of water, the initial crude ethanol product stream may be fed through a first water permeable membrane. Then, the retentate stream may be fed through a second water permeable membrane to yield a second retentate stream. The second permeate stream may be recycled and combined with the initial crude ethanol product stream to capture additional ethanol.

Water stream 183 may be fed through a second water permeable membrane 181 to generate a second retentate stream 184 and a second water stream 185. The second water stream 185 has a higher concentration of water than water stream 183. The second water stream 185 may be removed and discarded from the system. The second retentate stream 184 comprises ethanol, acetic acid and ethyl acetate, and may be combined with the first retentate stream 182 and jointly introduced to acid separation column 130.

As discussed above in connection with FIGS. 2-4, column 130 is an acid separation column. Column 130 is used to separate the retentate streams 182 and 184 into a residue stream 131 that comprises acetic acid and a distillate that comprises ethanol and ethyl acetate. In acid separation column 130, unreacted acetic acid and other heavy components, if present, are removed from the first and second retentate streams 182 and 184 and are withdrawn, preferably continuously, as residue 131. The unreacted acetic acid in residue stream 131 may be fed to vaporizer 110 as starting material to generate more ethanol. Column 130 also forms an overhead distillate, which is withdrawn in line 132, and which may be condensed and refluxed, for example, at a ratio of from 10:1 to 1:10, e.g., from 3:1 to 1:3 or from 1:2 to 2:1. The distillate in line 132 preferably comprises ethanol, ethyl acetate, and small amount of water, along with other impurities, which may be difficult to separate due to the formation of binary and tertiary azeotropes. Distillate 132 is compressed and fed to ethanol permeable membranes 170 and 171 to separate ethanol and ethyl acetate as discussed above in FIG. 7. Again, ethyl acetate permeable membranes may be substituted for ethanol permeable membranes 170 and 171.

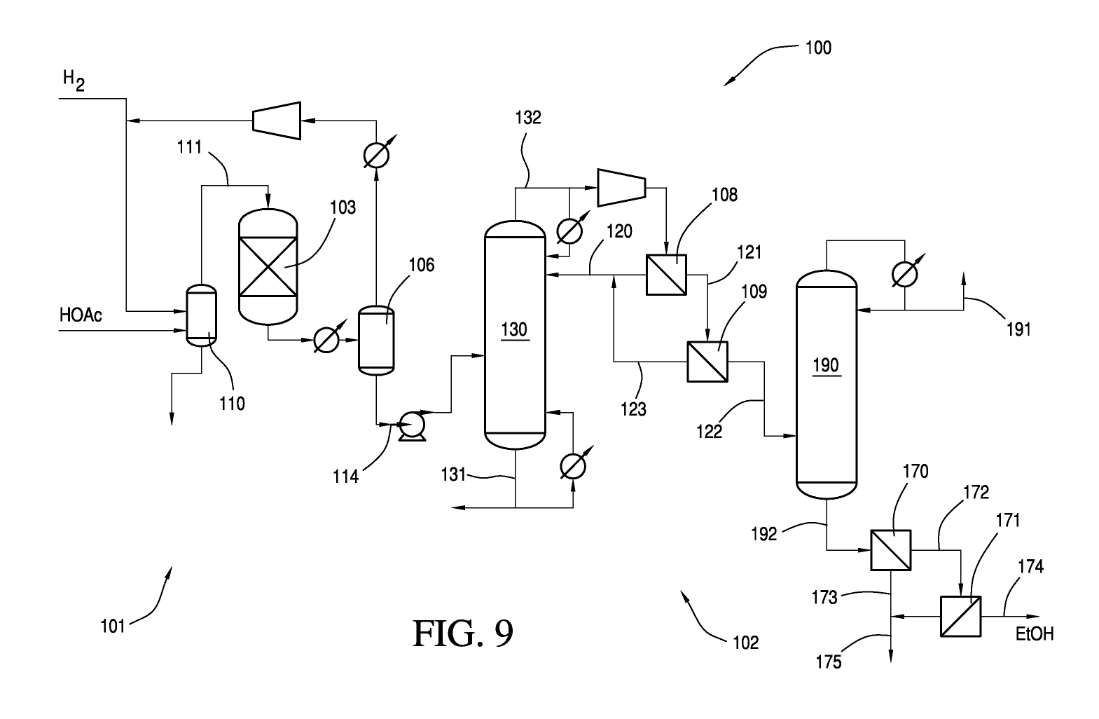

FIG. 9 shows a separation zone 102 with an acid separation column 130, water permeable membranes 108 and 109, an acetaldehyde removal column 190, and ethanol permeable membranes 170 and 171. Crude ethanol product in line 114 is introduced to distillation column 130 and separated into a residue stream 131 comprising acetic acid and a distillate stream 132 comprising ethanol, ethyl acetate, acetaldehyde, and water, as discussed in FIG. 2. The distillate stream 132 is optionally compressed and fed through water permeable membranes and 109 to remove water, as discussed above. The column may run at high enough pressure to facilitate membrane separation. Resulting water streams 120 and 123 may be combined with the reflux of distillate 132 and fed to first column 130.

In some embodiments, the amount of ethyl acetate may be greater making it is desirable to recover both an ethanol product and an ethyl acetate product. As shown in FIG. 9, the ethanol product stream 122, which comprises ethanol, ethyl acetate and acetaldehyde, is introduced to an acetaldehyde removal column 190. In column 190, the ethanol product stream 122 is separated into a distillate 191 that comprises acetaldehyde and a residue 192 that comprises ethyl acetate and ethanol. The distillate preferably is refluxed at a reflux ration of from 1:20 to 20:1, e.g., from 1:15 to 15:1 or from 1:10 to 10:1, and a portion of the distillate 191 may be returned to the reaction zone 101. In one embodiment, the distillate 191 may be combined with the acetic acid feed line and co-fed to vaporizer 110 to generate more ethanol product.

Acetaldehyde removal column 190 is preferably a tray column as described above and preferably operates above atmospheric pressure. In one embodiment, the pressure is from 120 kPa to 5,000 kPa, e.g., from 200 kPa to 4,500 kPa, or from 400 kPa to 3,000 kPa. In a preferred embodiment, the column 190 operates at a pressure that is higher than the pressure of the other columns. The temperature of the distillate exiting in line 191 from acetaldehyde removal column 190 preferably is from 60° C. to 110° C., e.g., from 70° C. to 100° C. or from 75° C. to 95° C. The temperature of the residue exiting in line 192 preferably is from 70° C. to 115° C., e.g., from 80° C. to 110° C. or from 85° C. to 110° C. Exemplary components of the distillate and residue compositions for acetaldehyde removal column 190 are provided in Table 5 below. It should be understood that the distillate and residue may also contain other components, not listed in Table 5.

TABLE 5
ACETALDEHYDE COLUMN 190

| | Conc. (wt. %) | Conc. (wt. %) | Conc. (wt. %) |
|---|---|---|---|
| Distillate | | | |
| Acetaldehyde | 2 to 90 | 2 to 50 | 5 to 40 |
| Ethyl Acetate | <90 | 30 to 80 | 40 to 75 |
| Ethanol | <30 | 0.001 to 25 | 0.01 to 20 |
| Water | <25 | 0.001 to 20 | 0.01 to 15 |
| Residue | | | |
| Ethyl Acetate | 40 to 100 | 50 to 100 | 60 to 100 |
| Ethanol | <40 | 0.001 to 30 | 0 to 15 |
| Water | <25 | 0.001 to 20 | 2 to 15 |
| Acetaldehyde | <1 | 0.001 to 0.5 | Not detectable |
| Acetal | <3 | 0.001 to 2 | 0.01 to 1 |

Residue 192 comprises ethanol and ethyl acetate and may be separated using ethanol permeable membranes 170 and 171, as described above in connection with FIGS. 7 and 8. Preferably, second ethyl acetate stream 175 is recovered as a separate product and is not returned to the reaction zone 101. Again, ethyl acetate permeable membranes may be substituted for ethanol permeable membranes 170 and 171

Membrane Separation Systems without Columns

FIGS. 10 and 11 are membrane separation systems 200 that use gas phase separation with membranes and without using distillation columns. Hydrogen in feed line 201 and acetic acid in feed line 202 are directed to a vaporizer 203 to create a vapor feed stream in line 204. The temperature of the vapor feed stream in line 204 is preferably from 100° C. to 350° C., e.g., from 120° C. to 310° C. or from 150° C. to 300° C. Vapor feed stream in line 204 is directed to the top of reactor 205. In addition, although FIGS. 10 and 11 shows line 204 being directed to the top of reactor 205, line 204 may be directed to the side, upper portion, or bottom of reactor 205. Reactor 205 is preferably similar to the reactor described above in FIG. 1.

During the hydrogenation process, a crude ethanol product stream is withdrawn, preferably continuously, from reactor 205 via line 206. Crude ethanol product stream 206 is fed to water permeable membranes 207 and 208 in FIG. 10. Water permeable membrane 207 has a selectivity for water and separates a water permeate stream 209 and a retentate stream 210. Retentate stream 210 preferably comprises ethanol and a minor portion of water. Retentate stream 210 is fed to a second water permeable membrane 208, which also has a higher selectivity to water. The retentate stream 211 of water permeable membrane 208 comprises ethanol and is condensed as the final product. The water permeate stream 212 of membrane 208 is co-fed with crude ethanol product stream 206 and fed to water permeable membrane 207. Optionally one or more water permeate streams 212 may pass through one or more compressors before being introduced to membrane 207.

The water permeate stream 209 of water permeable membrane 207 is condensed and fed to flasher 213. In one embodiment, any light gas, such as hydrogen, may pass through water permeable membrane 207 with the water in the water permeate stream 209. Flasher 213 operates at conditions sufficient to provide a vapor stream 214 and a liquid stream 215. Vapor stream 214 may comprise hydrogen and hydrocarbons, which may be purged and/or returned to reactor 205. The vapor stream 214 passes through compressor 216 and is combined with the hydrogen feed and co-fed to vaporizer 203.

In FIG. 11, crude ethanol product stream 206 is fed to membranes 220, 221 and 222. Hydrogen permeable membrane 220 has a selectivity for hydrogen. Acid permeable membrane 221 has a selectivity for acetic acid. Water permeable membrane 222 has a selectivity for water. Crude ethanol product stream 206 passes through hydrogen permeable membrane 220 to remove hydrogen as a permeate stream 223 and forms first intermediate retentate stream 224. Hydrogen permeate stream 223 may be returned to the reactor by passing through compressor 216. Optionally, permeate stream 223 may be superheated prior to passing through compressor 216 to ensure that only gases and vapors go through the compressor. A portion of retentate stream 224 is fed to acetic acid membrane 221. Membrane 221 separates an acetic acid permeate stream 226 and forms a second intermediate retentate stream 225. Acetic acid permeate stream 226 may be directed to reactor by co-feeding with acetic acid feed stream 202. Acetic acid permeating membrane has low selectivity generally. Multiple membranes will be needed. The retentate stream 225 of acetic acid membrane 221 is withdrawn and fed to water permeable membrane 222. In some embodiments, a portion of the first intermediate retentate stream 224′ may be fed to the water permeable membrane 222. Water permeable membrane 222 separates an ethanol and ethyl acetate retentate stream 227 and a water permeate stream 228. The ethanol will further be separated from ethyl acetate in another ethanol-permeating membrane.

### Ethanol Compositions

The finished ethanol composition obtained by the processes of the present invention preferably comprises from 75 to 96 wt. % ethanol, e.g., from 80 to 96 wt % or from 85 to 96 wt. % ethanol, based on the total weight of the finished ethanol composition. Exemplary finished ethanol compositional ranges are provided below in Table 6.

TABLE 6
FINISHED ETHANOL COMPOSITIONS

| | Conc. | | |
|---|---|---|---|
| Component | (wt. %) | Conc. (wt. %) | Conc. (wt. %) |
| Ethanol | 75 to 96 | 80 to 96 | 85 to 96 |
| Water | <12 | 1 to 9 | 3 to 8 |
| Acetic Acid | <1 | <0.1 | <0.01 |
| Ethyl Acetate | <2 | <0.5 | <0.05 |
| Acetal | <0.05 | <0.01 | <0.005 |
| Acetone | <0.05 | <0.01 | <0.005 |
| Isopropanol | <0.5 | <0.1 | <0.05 |
| n-propanol | <0.5 | <0.1 | <0.05 |

The finished ethanol composition of the present invention preferably contains very low amounts, e.g., less than 0.5 wt. %, of other alcohols, such as methanol, butanol, isobutanol, isoamyl alcohol and other C₄-C₂₀ alcohols. In one embodiment, the amount of isopropanol in the finished ethanol composition is from 80 to 1,000 wppm, e.g., from 95 to 1,000 wppm, from 100 to 700 wppm, or from 150 to 500 wppm. In one embodiment, the finished ethanol composition is substantially free of acetaldehyde, optionally comprising less than 8 wppm acetaldehyde, e.g., less than 5 wppm or less than 1 wppm.

In some embodiments, when further water separation is used, the ethanol product may be withdrawn as a stream from the water separation unit such as adsorption units, membranes, molecular sieves, extractive distillation units, or a combination thereof. In such embodiments, the ethanol concentration of the ethanol product may be higher than indicated in Table 7, and preferably is greater than 97 wt. % ethanol, e.g., greater than 98 wt. % or greater than 99.5 wt. %. The ethanol product in this aspect preferably comprises less than 3 wt. % water, e.g., less than 2 wt. % or less than 0.5 wt. %.

The finished ethanol composition produced by the embodiments of the present invention may be used in a variety of applications including applications as fuels, solvents, chemical feedstocks, pharmaceutical products, cleansers, sanitizers, hydrogenation transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, aldehydes, and higher alcohols, especially butanol. In the production of ethyl acetate, the finished ethanol composition may be esterified with acetic acid. In another application, the finished ethanol composition may be dehydrated to produce ethylene. Any known dehydration catalyst can be employed to dehydrate ethanol, such as those described in copending U.S. Pub. Nos. 2010/0030002 and 2010/0030001, the entire contents and disclosures of which are hereby incorporated by reference. A zeolite catalyst, for example, may be employed as the dehydration catalyst. Preferably, the zeolite has a pore diameter of at least about 0.6 nm, and preferred zeolites include dehydration catalysts selected from the group consisting of mordenites, ZSM-5, a zeolite X and a zeolite Y. Zeolite X is described, for example, in U.S. Pat. No. 2,882,244 and zeolite Y in U.S. Pat. No. 3,130,007, the entireties of which are hereby incorporated herein by reference.

In order that the invention disclosed herein may be more efficiently understood, an example is provided below. It should be understood that this example is for illustrative purposes only and is not to be construed as limiting the invention in any manner.

### Example

Acetic acid was hydrogenated in the presence of a catalyst with a conversion rate of 90.0%. Crude ethanol product stream having 52.4 wt. % ethanol, 24.6 wt. % water, 13.2 wt. % acetic acid, 8.5 wt. % of ethyl acetate and 0.6 wt. % acetaldehyde was fed to an acid separation column. The distillate stream contained 74.4 wt. % ethanol, 12.1 wt. % ethyl acetate, and 11.8 wt. % water. The residue stream comprised 44.6 wt. % acetic acid, and 55.4 wt. % water.

The distillate stream of the acid separation column fed to a light ends column. The distillate stream contained 79.5 wt. % ethyl acetate, 8.7 wt. % water, 0.4 wt. % ethanol, and 5.8 wt. % acetaldehyde. The residue stream comprised 28.7 wt. % ethanol, and 70.9 wt. % water. The light ends column was an extractive column and water is fed as an extractive agent.

The residue stream of the light ends column is fed to array of membranes having a selectivity for water. The permeate stream contained 94.9 wt. % ethanol and 4.0 wt. % water, and the retentate stream contained water in an amount greater than 99.9 wt. %. A portion of the retentate stream was returned to the light ends column as an extractive agent.

While the invention has been described in detail, modifications within the spirit and scope of the invention will be readily apparent to those of skill in the art. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing ethanol, comprising the steps of:
hydrogenating acetic acid in a reactor in the presence of a catalyst to form a crude ethanol product comprising ethanol, acetic acid and water;
separating at least a portion of the crude ethanol product in a distillation column into a distillate comprising ethanol and water, and a residue comprising acetic acid and water; and
passing at least a portion of the distillate stream to one or more membranes to yield an ethanol stream and a water stream.

2. The process of claim 1, wherein the crude ethanol product comprises from 15 wt. % to 70 wt. % of ethanol.

3. The process of claim 1, wherein the ethanol stream comprises ethanol in an amount greater than 85 wt. %.

4. The process of claim 1, further comprising returning a portion of the water stream to the distillation column.

5. The process of claim 1, further comprising separating a portion of the residue to yield an acetic acid stream that is recycled to the reactor.

6. The process of claim 1, further comprising the steps of:
separating at least a portion of the crude ethanol product in one or more membranes having a selectivity for hydrogen to yield an hydrogen stream and a retentate stream of the crude ethanol product;
returning the hydrogen stream to the reactor; and
introducing at least a portion of the retentate stream to the distillation column.

7. The process of claim 1, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

8. A process for producing ethanol, comprising the steps of:
hydrogenating acetic acid in a reactor in the presence of a catalyst to form a crude ethanol product comprising ethanol, ethyl acetate, and acetic acid;
separating at least a portion of the crude ethanol product in a distillation column into a distillate comprising ethanol and ethyl acetate, and a residue comprising acetic acid; and
passing at least a portion of the distillate stream to one or more membranes to yield an ethanol stream and an ethyl acetate stream.

9. The process of claim 8, wherein the one or more membranes have a selectivity for ethanol.

10. The process of claim 8, further comprising introducing a portion of the ethyl acetate stream to the reactor.

11. The process of claim 8, further comprising the steps of:
separating at least a portion of the crude ethanol product in one or more membranes having a selectivity for water to yield an water stream and a retentate stream of the crude ethanol product; and
introducing at least a portion of the retentate stream to the distillation column.

12. The process of claim 8, further comprising the steps of:
separating at least a portion of the crude ethanol product in one or more membranes having a selectivity for hydrogen to yield an hydrogen stream and a retentate stream of the crude ethanol product;
returning the hydrogen stream to the reactor; and
introducing at least a portion of the retentate stream to the distillation column.

13. A process for producing ethanol, comprising the steps of:
hydrogenating acetic acid in a reactor in the presence of a catalyst to form a crude ethanol product comprising ethanol, ethyl acetate, water, and acetic acid;
separating at least a portion of the crude ethanol product in a first distillation column into a first distillate comprising ethanol, ethyl acetate, and water, and a first residue comprising acetic acid; and
separating at least a portion of the first distillate in a second distillation column into a second distillate comprising ethyl acetate, and a second residue comprising ethanol and water; and
passing at least a portion of the second residue to one or more membranes to yield an ethanol stream and an water stream.

14. A process for producing ethanol, comprising the steps of:
hydrogenating acetic acid in a reactor in the presence of a catalyst to form a crude ethanol product, comprising ethanol, acetic acid, and water;
passing at least a portion of the crude ethanol product to a first membrane to separate a first permeate stream comprising acetic acid and a first retentate stream comprising ethanol and water;
passing the first retentate stream to a second membrane to separate a second permeate stream comprising water and a second retentate stream comprising ethanol.
